(19) United States
(12) Patent Application Publication
(10) Pub. No.: US 2010/0185294 A1
(43) Pub. Date: Jul. 22, 2010

(54) Nanorough Alloy Substrate

(75) Inventor: Steven W. Ek, Bolton, MA (US)

Correspondence Address: GROSSMAN, TUCKER, PERREAULT & PFLEGER, PLLC, 55 SOUTH COMMERICAL STREET, MANCHESTER, NH, 03101, US

(73) Assignee: ARTHROSURFACE INCORPORATED, Franklin, MA (US)

(21) Appl. No.: 12/711,039

(22) Filed: Feb. 23, 2010

Related U.S. Application Data
(63) Continuation-in-part of application No. 12/620,309, filed on Nov. 17, 2009.
Application No. 12/620,309 is a continuation of application No. 10/760,965, filed on Jan. 20, 2004, now Pat. No. 7,618,462.
Application No. 10/760,965 is a continuation of application No. 10/162,533, filed on Jun. 4, 2002, now Pat. No. 6,679,917.
(60) Provisional application No. 61/154,608, filed on Feb. 23, 2009.
(60) Provisional application No. 61/154,829, filed on Feb. 24, 2009.

(51) Int. Cl. A61F 2/30

(52) U.S. Cl. 623/18.11

(57) ABSTRACT

A CoCr substrate for growing mammalian cells and, in particular, chondrocytes and synovial stem cells. The substrate may be treated electrolytically to produce a surface topography that is amenable to cell growth and migration. The substrate shows improved adhesion and migration characteristics and may be useful as a prosthesis or implant and in particular as a prosthesis in a joint such as the knee.

### RELATED APPLICATIONS

This application claims benefit of U.S. Provisional Patent Application Ser. No. 61/154,608, titled NANOROUGH ALLOY SUBSTRATE, filed Feb. 23, 2009 and of U.S. Provisional Patent Application Ser. No. 61/154,829, titled NANOROUGH ALLOY SUBSTRATE, filed Feb. 24, 2009. This application is also a Continuation in Part of pending U.S. patent application Ser. No. 12/620,309 filed Nov. 17, 2009 which is a continuation application of U.S. application Ser. No. 10/760,965 filed Jan. 20, 2004, now U.S. Pat. No. 7,618,462, which is a continuation of U.S. application Ser. No. 10/162,533 filed Jun. 4, 2002, now U.S. Pat. No. 6,679,917. Each of these provisional and non-provisional applications is hereby incorporated by reference herein.

### BACKGROUND

1. Field of Invention

The invention relates to surfaces for growing cells and, in particular, to substrates suitable for supporting chondrocytes and synovial stem cells.

2. Discussion of Related Art

Joint disease or injury can sometimes be controlled, cured or ameliorated through the use of implants or prostheses. These implants or prostheses may be made of biocompatible materials that can be used to supplement or replace tissue such as bone and/or cartilage. In some cases, these materials may form a substrate on which cells, such as bone and/or cartilage cells, can adhere and/or grow.

### SUMMARY

The subject matter of this application may involve, in some cases, interrelated products, alternative solutions to a particular problem, and/or a plurality of different uses of a single system or article.

In one aspect, a joint prosthesis is provided, the joint prosthesis including a first surface having a curvature that substantially matches the contour of native articular surface, said first surface comprising a cobalt chromium alloy having an average surface feature size of between 10 and 30 nm.

In another aspect, a prosthesis for supporting the growth of mammalian cells is provided, the prosthesis comprising a cobalt chromium alloy surface having a surface energy of greater than 30 mJ/m².

In another aspect, a prosthesis for supporting mammalian cells is provided, the prosthesis comprising a contoured surface comprising a cobalt chromium alloy exhibiting a wet contact angle of less than about 60 degrees.

### BRIEF DESCRIPTION OF THE DRAWINGS

FIG. 1 is a bar graph illustrating test results regarding chondrocyte adhesion;

FIG. 2 is a bar graph illustrating test results regarding synovial stem cell adhesion;

FIG. 3 is a bar graph illustrating test results for regarding chondrocyte migration;

FIG. 4 is a bar graph illustrating test results regarding synovial stem cell migration;

FIG. 5 is a bar graph illustrating test results regarding chondrocyte GAG synthesis;

FIG. 6 is a bar graph illustrating test results regarding synovial stem cell GAG synthesis;

FIG. 7 is a bar graph illustrating test results regarding chondrocyte collagen synthesis;

FIG. 8 is a bar graph illustrating test results regarding synovial stem cell collagen synthesis;

FIG. 9 is a bar graph illustrating test results regarding adsorption of fibronectin, vitronectin and IgG;

FIG. 10 is a bar graph illustrating test results regarding cell binding regions for RGD and Heparin Sulfate; and

FIG. 11 is a bar graph illustrating cell density test results for four different CoCr substrates.

FIG. 12 is a photocopy of a micro CT scan (coronal plane) of an implant including a treated CoCr outer surface.

FIG. 13 is a photocopy of a micro CT scan (sagittal plane) of an implant including a treated CoCr outer surface.

FIG. 14 is a photograph illustrating a manufactured condyle defect in a subject animal.

FIG. 15 is a photograph illustrating the surface of an untreated implant after 26 weeks.

FIG. 16 is a photograph illustrating the surface of a treated implant after six weeks.

FIG. 17 is a photograph illustrating the tissue growth over the surface of a treated implant after 12 weeks.

### DETAILED DESCRIPTION

In one aspect a metallic substrate can be used as a surface for supporting mammalian cells such as chondrocytes and synovial stem cells. The substrate may be capable of being inserted into one or more surfaces of a mammalian joint and may form part of, or an entire prosthesis. The prosthesis may include a threaded portion that is made from the substrate material or from a different material. For instance, the prosthesis may have a surface portion of CoCr and a threaded portion of CoCr or Ti. The substrate can include a metal or metal alloy such as, for example, cobalt chromium (CoCr). In some embodiments the CoCr may be doped with, for example, molybdenum (CoCrMo). In additional embodiments the alloy may also include zirconium. The substrate may be initially formed using methods such as casting or sintering. The material may be a unitary, continuous substrate or may be a coating on an alternative material. For example, a CoCr allow may be affixed to a titanium screw. The treated surface may be shaped to match the contour of the area in which it is being used. For instance, a CoCr cap may have a hemispherical surface that substantially matches the contour of the cartilage that it is implanted in. The surface of the substrate may include very small features on a nanometer scale. For example, the substrate may include surface features that are less than 50 nm, less than 40 nm or less than 30 nm in size, and may be greater than 10 or greater than 20 nm in size. The entire surface of the substrate may be treated, or treatment may be limited to particular surfaces, such as those where cell growth is to be promoted. In some embodiments, the average dimension of the surface features may be between 5 and 30 nm, between 10 and 30 nm or between 15 and 25 nm when measured using Atomic Force Microscopy (AFM). The surface may also exhibit improved wettability as evidenced by a contact angle of less than 50°, less than 40°, less than 30°, less than 20° or less than 15°. Surface energy may be greater than with conventional CoCr surfaces and in some embodiments may be greater than 12 mJ/m², greater than 20 mJ/m², greater than 30 mJ/m² or greater than 40 mJ/m². These features may provide for improved cell (e.g., chondrocyte) adhesion and/or growth on the substrate. Cells may be grown directly on the surface of the treated alloy in the absence of any other coating material. The features can be formed on the surface of the substrate by passing current through the material (as the positive electrode) in an acidic electrolyte solution such as 1 M H₂SO₄. The surface of the material may be considered to be “anodized” after this procedure.

In another aspect, a method is provided for preparing a material as a substrate for cell adhesion and/or growth. The material may be a metal such as a metallic alloy. Preferred alloys include CoCr which may include or be void of Mo, Ta and/or W. The CoCr may include one or more of carbon, molybdenum and nitrogen. In some embodiments, the composition of the CoCr may include (by weight) 0.01 to 1.0% C, 20 to 40% Co, 1 to 10% Mo, 0.01 to 1.0% N and the balance Co. In one set of embodiments the CoCr alloy includes 0.2 to 0.3% % C, 26 to 30% Co, 5 to 7% Mo, 0.15 to 0.2% N and the balance Co. An example of material that has been shown to be useful is BioDur® CCM Plus® Alloy, available from Carpenter Specialty Alloys. Other preferred metals include, for example, titanium, which may be treated in the same manner using the methods described herein. In some embodiments, the material may consist essentially of CoCr. The material may be prepared so that it exhibits a surface structure that is amenable to the adhesion and growth of mammalian cells such as chondrocytes and synovial stem cells. The material may include surface features as described herein. The material may then be provided for implantation into a mammalian subject and may be promoted for such uses.

The treatment procedure can include an electrolytic process that may be referred to herein as anodization, but it is understood that any chemical transformation on the surface of the material may not be identical to that achieved via traditional anodization, such as when aluminum is anodized. The material being prepared may be configured as the anode (positive electrode) in an electrolytic process. The cathode may be of any appropriate material, for example, a precious metal such as platinum. The anode material may be placed in an acidic solution such as a mineral acid. Examples of specific acids that may be used include, but are not limited to, chromic acid, hydrofluoric acid, nitric acid, sulfuric acid, phosphoric acid or an organic acid. The solution may be contained in a glass or polymeric vessel suitable for use with the chosen electrolyte. In one set of embodiments, the electrolyte is sulfuric acid. The concentration of the acid solution may be, for example, between 0.1 and 5 M, between 0.5 and 2 M, or about 1.0 M. In one embodiment, 1.0 M sulfuric acid is used. In some embodiments the applied voltage can be less than that used in standard anodization processes. For instance, the voltage may be less than 20 V. Ranges for the applied voltage may be, for example, greater than 100 mV and less than 30 V. Alternative voltage ranges include 100 mV to 10 V, 500 mV to 5 V, 1 V to 3 V and 2 V. The voltage may be applied for a time sufficient to achieve the desired surface effect. In some cases this may be less than one hour, less than 10 minutes, less than 5 minutes or less than two minutes. In other embodiments voltage may be applied for more than 10 seconds, more than 30 seconds or more than one minute. In one specific set of embodiments a charge of two volts is applied for a period of two minutes. During the electrolytic procedure the solution may be agitated by, for example, using magnetic agitation.

The substrate surface resulting from the above procedure can include unique surface characteristics that make it ideal for cell adhesion and growth. For instance, the surface may provide for improved adhesion and growth of chondrocytes and/or synovial stem cells. These features may be between 10 and 30 nm in size and, in some cases, may be between 20 and 25 nm in size. The features may be substantially spherical, meaning that the features are substantially in the shape of a portion of a sphere that extends outwardly from the surface of the substrate. The features may be equally and randomly spaced from each other. The features may be detectable using Atomic Force Microscopy (AFM) set to scan at 1 μm by 1 μm, which, for one set of embodiments, has resulted in RMS values of about 23.5 nm. Thus, the feature size for the treated CoCr is about 20 nm while for the untreated material there are no surface features (none detectable) observed at the nanometer level. The untreated samples are therefore considered to be “nano-smooth.” It is notable that when scanned at 5 μm by 5 μm that the RMS of features detected for both the untreated and treated material are about 50 nm. Similarly, at 25 μm by 25 μm, the RMS of features for both the treated and untreated CoCr are about 2 um.

Treatment using the electrolytic procedure described above can also alter the surface energy and the contact angle of the substrate. Regarding contact angle, untreated CoCr typically exhibits a contact angle of 65 degrees (measured using the method described below) while the same material, after electrolytic treatment, exhibits a contact angle of about 13 degrees. Similarly, surface energy can be increased from 12 mJ/m² to 45 mJ/m² after treatment

Experimental Results

A sample of CoCr (BioDur® CCM Plus® Alloy, including 0.2 to 0.3% C, 26 to 30% Cr, 5 to 7% Mo, 0.15 to 0.2% N and the balance Co) was treated by anodizing the material at a voltage of 2 volts for a period of 2 minutes in a 1 M H₂SO₄ solution. This is the same material used in all experiments herein unless otherwise specified. During anodization the electrolyte was agitated using a magnetic stirrer. Features and properties of the substrate were then characterized as provided below. Surface features were characterized using Atomic Force Microscopy (AFM).

Nanometer surface roughness measurements of both untreated and treated (as above) CoCr samples were performed using a multimode AFM (Dimension 3100, Veeco, Calif.). Scan areas of 1 μm×1 μm (nanoscale), 5 μm×5 μm (small micron scale), and 25 μm×25 μm (large micron scale) were used. Commercially available AFM tips (radius of tip curvature was less than 10 nm, NSC15/ALBS, Micro-Masch, OR) were used in tapping mode with a scan rate of 0.5 Hz. Full tip cone angles were 30° but less than 10° at the 200 nm tip apex (tip height was 25 μm with a constant force of 40 N/m). The exact nanometer surface feature for the samples was determined quantitatively from the 1 μm×1 μm AFM scan. The scans performed at 5×5 μm and at 25×25 μm showed little or no difference in features between the untreated and the treated sample. However, when the same samples were scanned at 1 μm×1 μm a significant change in the landscape was detected. While the untreated sample showed features of only about 1 nm, the treated samples were shown to have features of about 23.5 nm in size. Thus, although only detectable at the 1 μm×1 μm scale, this change in surface structure may be significant, and it is believed that this alteration provides a significantly improved cell substrate. This anodized material is referred to herein as “nanorough” material. Table 1 (below) provides data regarding the surface structure detected by AFM.

Contact angles and surface energy of the samples were also determined. A drop shape analysis system (Easy drop contact angle system, Kruss, Germany) with analysis software (DSA1) was used to determine the surface contact angles on the samples. Distilled water was used as the contacting solvent. All data were obtained five seconds after placing the droplet on the surfaces under ambient conditions. For the surface energy calculation, Eₛ=Eₗᵥ·cos θ was used where Eₗᵥ=72.8 mJ/m² at 20° C. for pure water and θ was the static contact angle. Here, Eₛ was the surface energy of the contacting surface and Eₗᵥ was the surface energy between the water and air under ambient conditions. Results are provided below in Table 1 and show that surface energy on the CoCr substrate increased from 12 mJ/m² on the untreated surface to 45 mJ/m² on the treated surface.

In between tests the samples were cleaned by soaking and sonicating separately in acetone and ethanol. The materials were then re-characterized for surface properties as described above and for chemistry using EDS (energy-dispersive spectroscopy, using an Oxford INCA 250 detector and associated software: Oxford Instrument America, Inc., Concord, Mass.). Differences between material characterization and cell/protein studies were determined using ANOVA followed by student t-tests.

TABLE 1
Material Characterization of CoCr Anodized and Unanodized Samples

| | AFM | | | | | |
|---|---|---|---|---|---|---|
| | RMS | AFM | AFM | | | |
| | (1 by | RMS | RMS | Average Surface | Contact | Surface |
| Sample | 1 μm | (5 by 5 μm | (25 by 25 μm | Feature Size | Angle | Energy |
| Treatment | Scan) | Scan) | Scan) | at the nm Level | (degrees) | (mJ/m²) |
| Untreated | 1.1 nm | 57 nm | 2.1 μm | Not Detectable | 65 | 12 |
| Electrolytically | 23.5 nm* | 50 nm | 1.9 μm | 20 nm diameter | 13* | 45* |
| Treated | | | | spheres* | | |

*p < 0.01 (compared to unanodized CoCr)

Additional experiments were designed and completed in order to assess the use of the nanorough CoCr material as a substrate for adhering and growing cells. The treated CoCr material was evaluated with both human articular chondrocytes and with synovial stem cells.

Human articular chondrocytes (cartilage-synthesizing cells obtained from Cell Applications Inc.) were cultured in Chondrocyte Growth Medium (Cell Applications Inc.) on 100 mm Petri dishes. The cells were incubated under standard cell culture conditions known to those skilled in the art, including a sterile, humidified, 5% CO₂, 95% air, 37° C. environment. Chondrocytes used were at passage numbers below 10. The phenotype of the chondrocytes had been previously characterized by the synthesis of Chondrocyte Expressed Protein-68 (CEP-68) for up to 21 days in culture under the same conditions as described above.

For a source of synovial stem cells, primary cells were isolated from the synovial membranes of 4 month-old female pig knee joints by mincing and enzymatic digestion, using 0.25% trypsin for 30 minutes and 0.4% collagenase II for one hour followed by filtration through 70 μm cell strainers. The cells were expanded in high-glucose DMEM (4.5 μg/L D-glucose, L-Glutamine, 1 mg/l sodium pyruvate) supplemented with 1.0% FBS, 1.0% ITS Premix, 1.00 U/ml penicillin, 1.00 pg/ml streptomycin, 2 mM L-glutamine and 2.5 μg/ml amphotericin B. The cells had been previously characterized. The same media was used for cell culture.

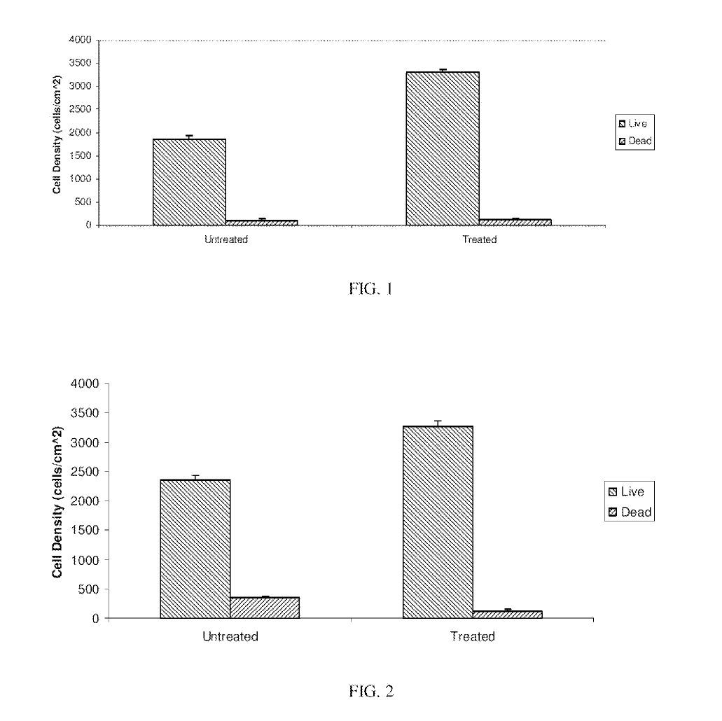

To characterize cell adhesion and density, the following experiments were performed on both the untreated and the treated (nanorough) material: Chondrocytes and synovial stem cells were separately seeded at 3,500 cells per square centimeter and were cultured for 1 hour. At the end of each time point, non-adherent cells were removed by rinsing with a phosphate buffered saline (PBS) solution while adherent cells were fixed, stained, and counted. For this purpose, the Live/Dead assay (Molecular Probes) was used for cell staining. Results comparing unanodized CoCr and treated CoCr are shown in bar graph form in FIG. 1 (chondrocytes) and FIG. 2 (synovial stem cells). These results indicate, surprisingly, that 100% of the cells adhered to the treated surface material. Less than two thirds of the cells adhered to the untreated CoCr substrate.

For the differentiation studies reported below, cells were seeded at 50,000 cells per square cm and were allowed to attach and grow for time points of 3, 5, and 7 days. At the end of each time point, non-adherent cells were removed by rinsing with phosphate buffered saline (PBS) solution. Cell counts at these long time periods were determined via the Cytotox 96 assay (Promega) according to the manufacturer's instructions. Specifically, substrates were placed into clean wells, frozen down at −70° C. for 30 min, and then incubated for 15 min at 37° C. to lyse the cells. The resulting solution was centrifuged at 250×g for 4 min and 50 μL of it was placed into a well of a 96-well plate. In total, 50 μL of Substrate Mix (Cytotox 96, Promega) was added and the plate was incubated for 30 min at room temperature, protected from light. After incubation, a Stop Solution (Cytotox 96, Promega) was added to each well and light absorbance was determined using a microplate reader and a spectrophotometer (SpectraMax 190, Molecular Devices Corp.) at 490 nm using computer software (SoftMax Pro 3.12, Molecular Devices Corp.). The resulting light absorbance was compared to a standard curve to calculate the number of cells. A standard curve was constructed by linear regression analysis, using light absorbance of cell lysates at known concentrations.

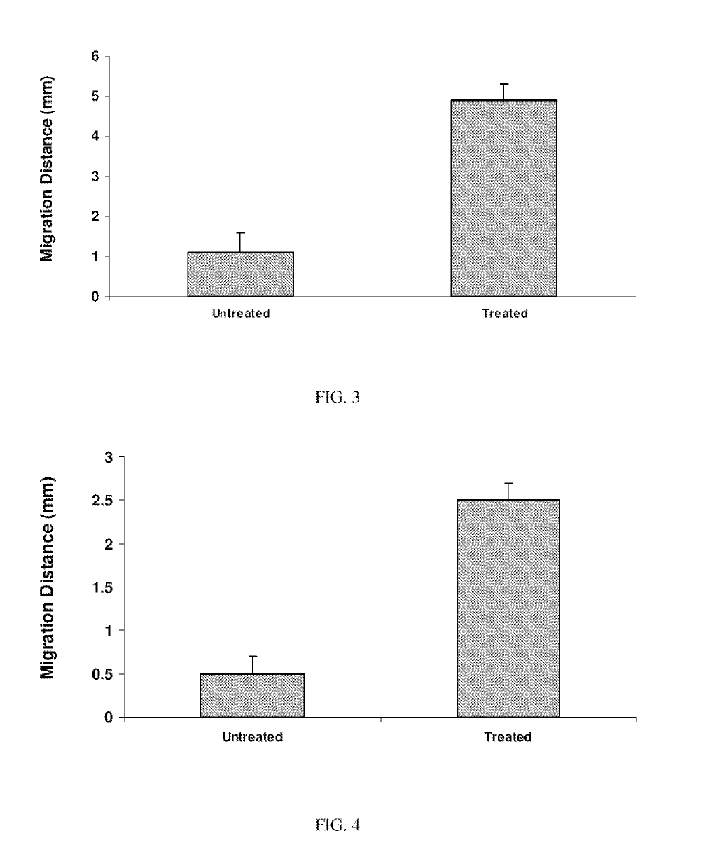

Cell migration (surface occupancy) was evaluated on both the treated and untreated CoCr samples. Sterile PTFE fences were placed on the treated and untreated CoCr substrates and covered the entire substrate surface except for a central circular well area. Cells (25,000 cells) were seeded into the central well of the fences in respective cell culture media and were allowed to adhere under standard cell culture conditions for 4 h. At that time, the fences were removed carefully (to avoid disturbing the adhering cells) and the medium in the wells was aspirated. The confluent cell colony was gently rinsed with phosphate-buffered saline (to remove non-adherent cells) and was then cultured in respective media under standard cell culture conditions for 4 days. Cells present on the substrates were then fixed in situ with 4% formaldehyde for 10 min, stained with Hoechst 33258, and visualized using fluorescence (365 nm excitation; 400 nm emission) microscopy. The total migration distance (expressed as mm) occupied by each cell colony was determined using Image Pro. Graphical results (mean+/−SEM; N=3; p<0.01(for treated compared to untreated)) of the study for chondrocytes are provided in FIG. 3, and results for synovial stem cells are provided in FIG. 4. Results for both cell types show an increase in migration of about 5× for the treated CoCr when compared to the untreated CoCr.

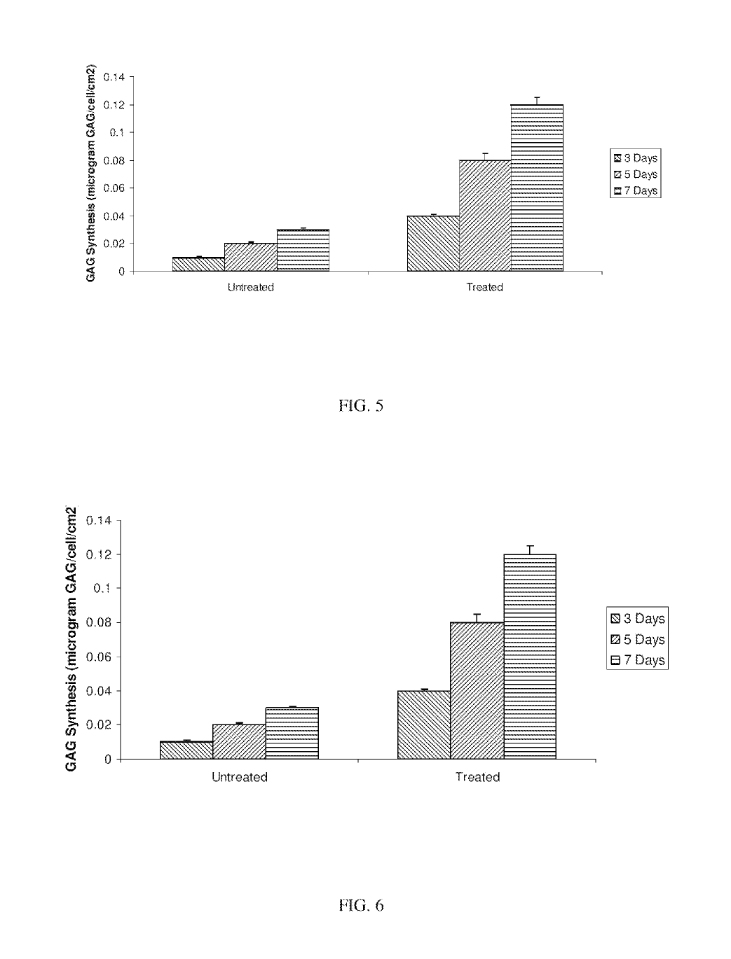

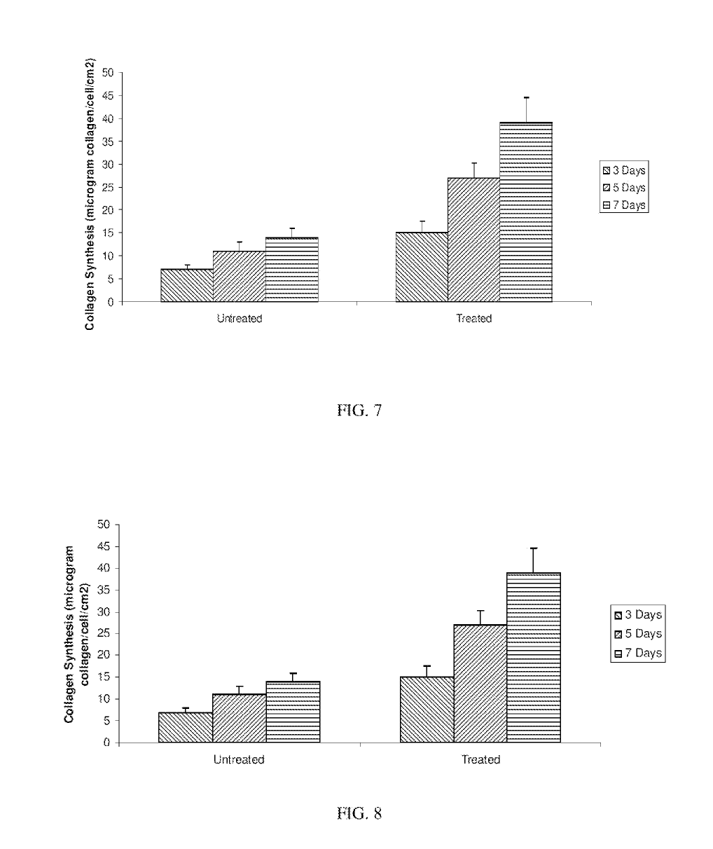

To determine the effect of substrate topology on cell differentiation an experiment was designed to measure some of the proteins (specifically, total intracellular collagen and glycosaminoglycans (GAGs)) synthesized by chondrocytes and synovial stem cells in situ. The amount of proteins produced was investigated as a measure of cell differentiation when cultured on the treated and untreated CoCr substrates. GAGs were determined using a commercially available spectrophotometer based assay (Blyscan assay kit, Biocolor) according to the instructions from the manufacturer. For this process, 50 μl aliquots of cell extracts obtained from cell lysates (prepared through three distilled water freeze-thaw cycles at the end of each time period) were placed into a set of 1.5 ml microcentrifuge tubes. The tubes were filled with PBS up to 100 μl. Blyscan dye (1 ml) was added to all tubes and the tubes were placed on a shaker for 30 min. At this point, tubes were centrifuged at 10,000×g for 10 min and the dye solution was carefully removed without disturbing the precipitated GAG on the bottom of the tube. Then, 1 ml of the dissociation reagent was added to the tubes and mixed well using a vortex mixer for 10 min. Each sample (200 μl) was transferred to a well in a 96-well plate and absorbance was read at 656 nm using a spectrophotometer. GAG content was normalized to cell number (determined as described above) and substrate surface area. For both the treated and untreated CoCr substrates, normalized GAG production results (mean+/−SEM; N=3; p<0.01) are provided graphically in FIG. 5 (chondrocytes) and FIG. 6 (synovial stem cells). At three days, five days and seven days the results show an increase in GAG synthesis of about 4× when comparing the treated CoCr to the untreated material.

Intracellular collagen concentrations were determined similarly using a Sirius Red dye stain (Direct Red; Sigma) and a spectrophotometer. Cells were lysed as above using freeze thaw methods. Specifically, the cell extracts (50 al well) were placed in 96-well plates in triplicate per substrate type. The plates were placed in a humidified incubator (at 37° C.) for 16 h and then in a dry incubator (at 37° C.) with desiccant. Each well was washed with 200 μl distilled water three times for a 1 min. wash. In each well, 100 μl of 0.1% Sirius Red stain (0.05 g Sirius Red powder per 50 ml picric acid) was allowed to sit for 1 h at room temperature. Using 200.1 of 0.1 M HCl, the plates were washed five times with a distilled water wash. The stain was then washed with 200 μl of 0.1 M NaOH for 5 min and was mixed well. The stain was placed into a second plate to read the absorbance in a microplate reader at 540 nm. A standard curve was constructed as micrograms of collagen versus absorbance at 540 nm. For the standard curve, 0.1% collagen type I solution (Sigma) was diluted at small increments and the light absorbance of the Sirius Red stain in these dilutions was recorded. The total amount of collagen was normalized to cell number and substrate surface area. Results are provided graphically in FIG. 7 (chondrocytes) and FIG. 8 (synovial stem cells) and indicate that at each of three days, five days and seven days the collagen synthesis, for both cell types, was more than double for the treated CoCr when compared to the untreated material.

To determine differences in initial protein interactions on the CoCr substrates, the protein binding characteristics of the substrates were evaluated. Test samples were separately soaked in 200 μl of the above described Chondrocyte Growth Media for 1 hour. The substrates were then rinsed with PBS, blocked with 2% bovine serum albumin (BSA; Sigma) for 1 h, and incubated with anti-bovine vitronectin (1:100; Accurate Chemical), anti-bovine fibronectin (1:100; Chemicon, Temecula, Calif.) and anti-IgG for 1 h (1:100 Chemicon). Immediately thereafter, the substrates were rinsed with Tris buffered saline-0.1%, Triton X-100 (Sigma) and incubated with horse radish peroxidase conjugated anti-rabbit secondary antibody (1:100; Bio-Rad). An ABTS (2,2′-azino-bis(3-ethylbenzthiazoline-6-sulfonic acid)) soluble substrate kit (Vector Labs, Burlingame, Calif.) was used to detect secondary antibodies spectrophotometrically (SpectroMAX 190, 488 nm; Molecular Devices, Palo Alto, Calif.) per the manufacturer's instructions. This was also completed using antibodies to arginine-glycine-aspartic acid (RGD; Molecular Probes) and heparin sulfate binding regions of proteins in order to ascertain bioactivity of the adsorbed proteins. Results providing relative elative adsorption of vitronectin, fibronectin and IgG are provided graphically in FIG. 9. Vitronectin and fibronectin adsorption results show an increase of greater than 10× when comparing the treated to the untreated CoCr. These two proteins are known to promote chondrocyte adhesion so these results indicate a significant improvement in chondrocyte adhesion with the treated CoCr. The adsorption of IgG, however, was much reduced on the treated CoCr. As IgG is known to initiate an inflammatory response, this reduction in IgG adhesion indicates that the treated CoCr is a superior substrate for growing chondrocytes, synovial stem cells and cartilage tissue.

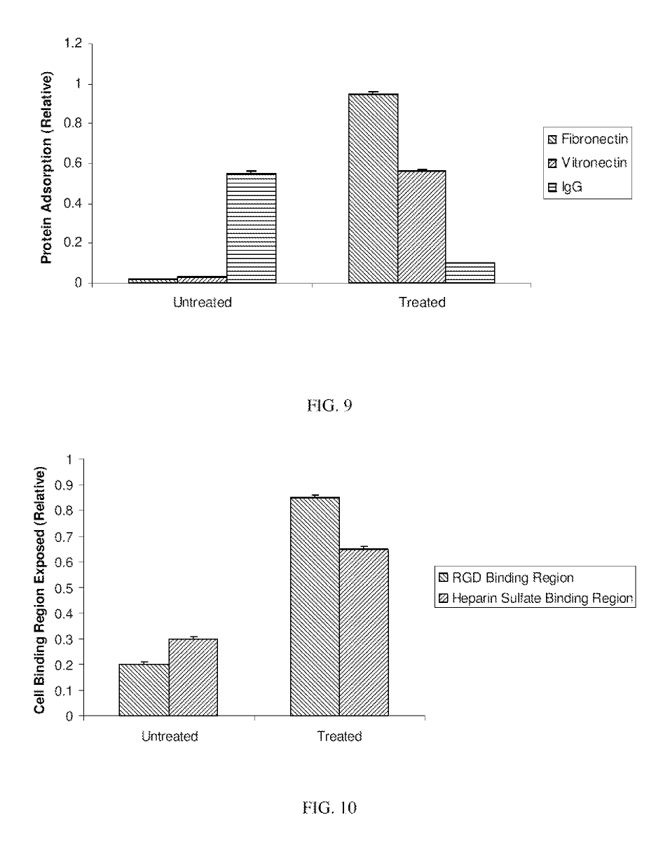

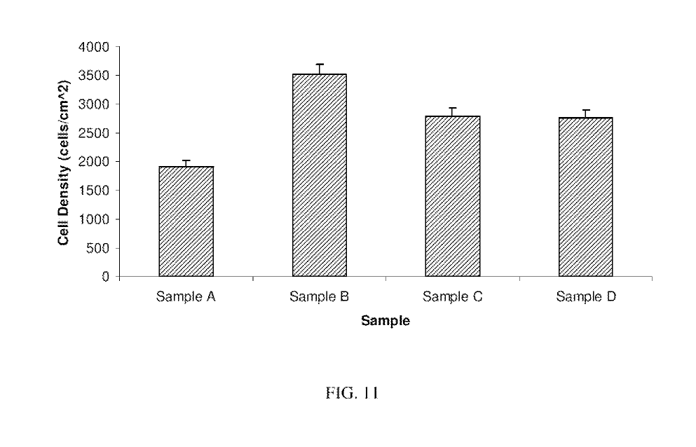

The results for the relative binding regions exposed for both RGD and heparin sulfate are provided graphically in FIG. 10. The treated CoCr realized a three to four fold increase in exposed binding regions for each of these proteins, indicating an improved ability to bind cells when compared to the untreated material.

In an additional experiment the CoCr substrate was anodized under various conditions by treating with three different electrolyte solutions. Sample A was untreated, sample B was treated in 1 M H₂SO₄, sample C was treated in 0.1 M H₂SO₄ and sample D was treated in 1 M H₃PO₄. Each sample was electrolytically treated for two minutes at a voltage of 2 V. Samples were then washed and evaluated for chondrocyte adhesion as outlined above. Results are provided graphically in FIG. 11 and indicate that the CoCr substrate treated in 0.1 M H₂SO₄ (sample C) and the substrate treated in 1 M H₃PO₄ (sample D) provided significantly improved chondrocyte adhesion than did the untreated sample (A). However, the CoCr treated in the 1 M H₂SO₄, provided a 100% adhesion rate which was not attained with the other substrates.

In an additional experiment, implants including the treated CoCr substrate were evaluated to see how the treated substrate compared to untreated substrate that had been tested previously. Each implant included a titanium screw portion for implantation into the bone. An exposed cap on each insert was made of a CoCr alloy. The treated implant was electrolytically treated as was the “electrolytically treated” sample in Table 1 above and exhibited the same surface characteristics as the electrolytically treated sample. The untreated sample which had been previously evaluated was identical to the untreated sample of Table 1. Each inserted implant was a size 12 cap with a 1.0 mm×1.5 mm offset. The devices were implanted using the standard clinical procedures developed by KirkerHead et al., and Walsh et al. One of the animals was sacrificed at 6 weeks after insertion and the other at 12 weeks.

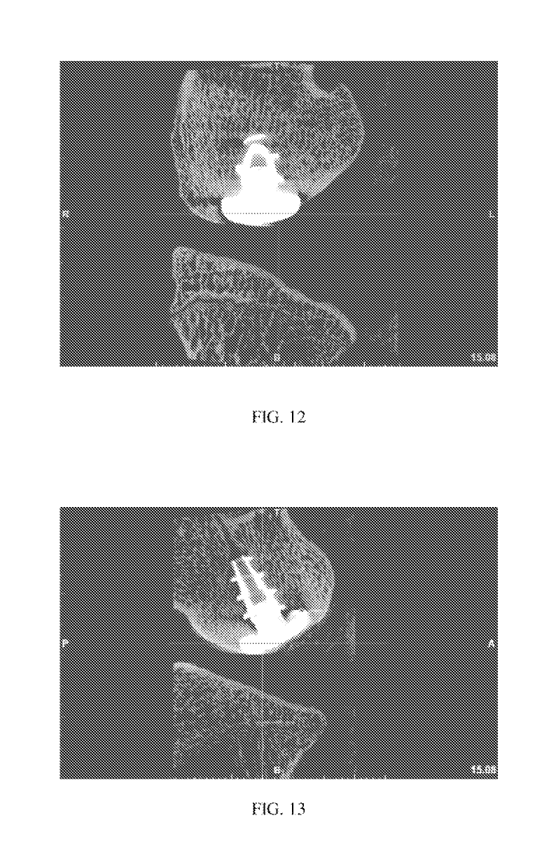

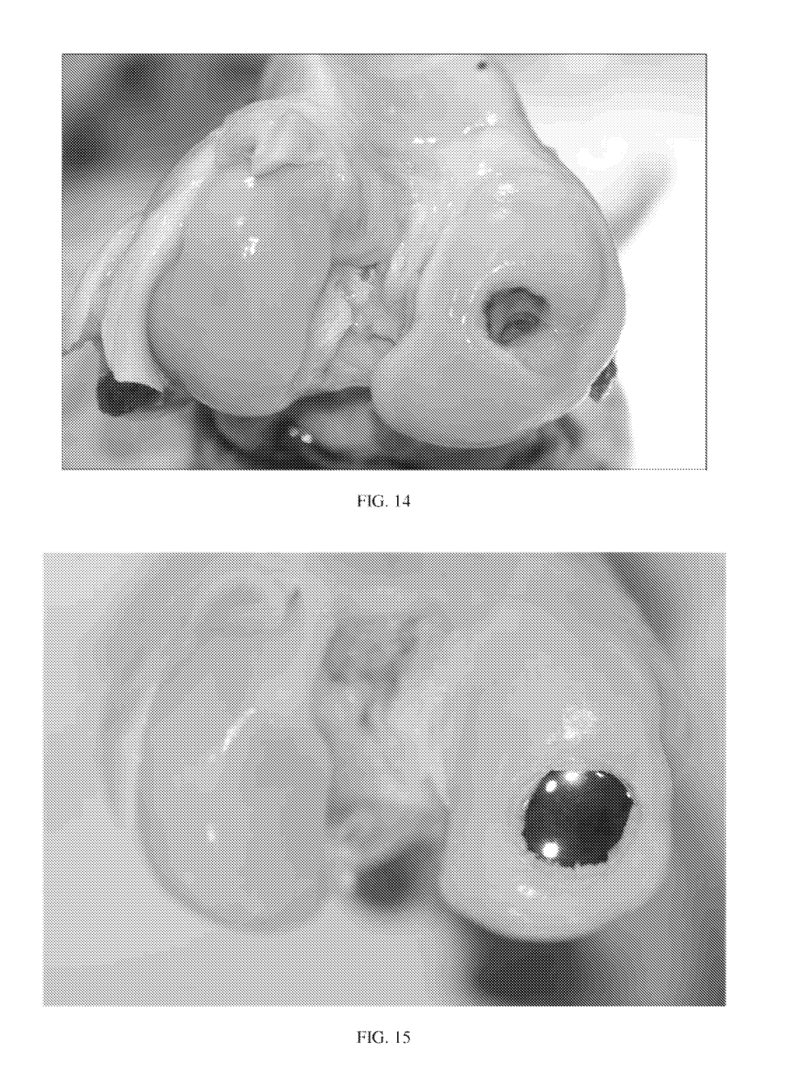

Each implant type was inserted into a rear femur of an anaesthetized two-year-old sheep after manufacturing a defect in the cartilage. The implants were inserted with the CoCr surface of the implant recessed slightly below the surface of the cartilage. Photocopies of micro computed tomography scans of the treated implant at six weeks are provided in FIG. 12 (coronal plane) and FIG. 13 (sagittal plane). The scans illustrate a threaded portion of the prosthesis inserted into the bone of the femur and a contoured surface portion that substantially matches the curvature of the native articular surface. A photograph of the manufactured defect prior to insertion of the implant is provided in FIG. 14. FIG. 15 is a photograph at 26 weeks of the lateral and medial femoral condyles including an untreated CoCr insert (right). The surface of the implant showed a lack of cell growth over the exposed region.

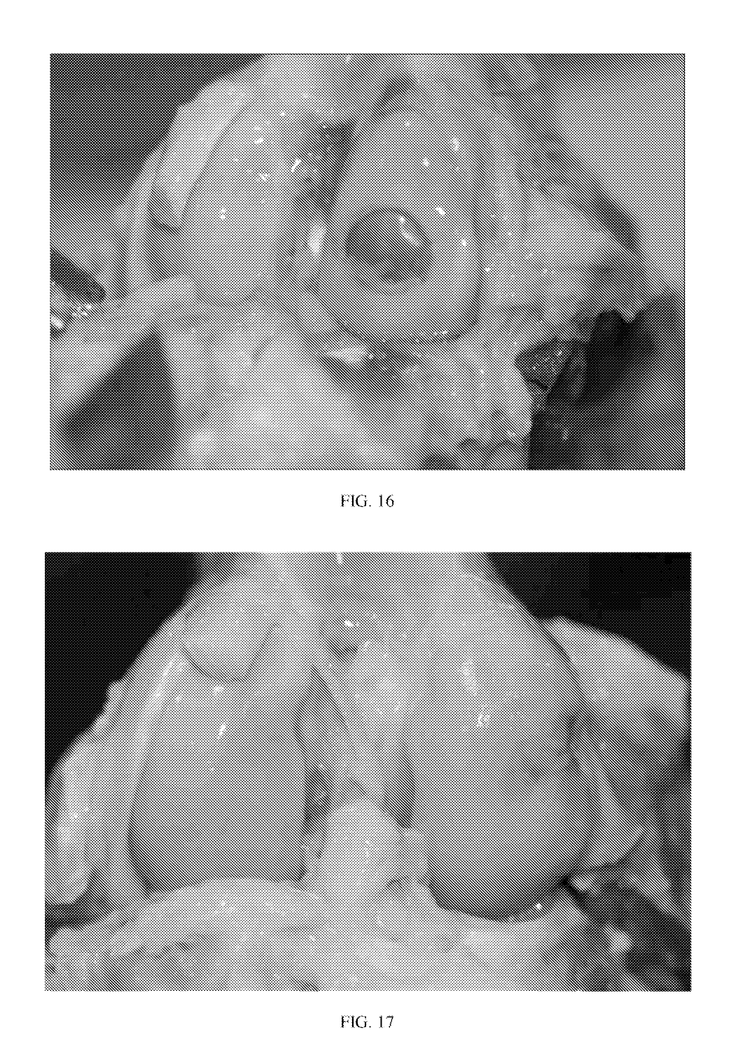

FIG. 16 is a photograph at 6 weeks of the lateral and medial femoral condyles including a treated CoCr insert (right). The photograph shows a thin film of cells over the entire exposed surface of the treated material.

FIG. 17 is a photograph at 12 weeks of the lateral and medial femoral condyles including a treated CoCr insert (right). The surface of the implant cannot be seen in the photograph and is completely covered by tissue.

A comparison of FIG. 15 (untreated) with FIGS. 16 and 17 provides visual evidence of vastly improved cell growth in vivo when the CoCr substrate is treated to produce a nanorough surface. The treated material, exhibiting a contact angle of 13 degrees, a surface energy of 45 mJ/m² and AFM RMS surface features of 23.5 nm (1 by 1 μm scan) provides for cell growth over the entire surface of the implant at six weeks and for extensive tissue growth over the implant at 12 weeks. This is in contrast to the untreated material exhibiting a contact angle of 65 degrees, a surface energy of 12 mJ/m² and AFM RMS surface features of 1.1 nm (1 by 1 μm scan) which showed an absence of cell growth after 26 weeks.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated in their entirety herein by reference.

What is claimed is:

1. A joint prosthesis comprising:
a first surface having a curvature that substantially matches the contour of native articular surface, said first surface comprising a cobalt chromium alloy having an average surface feature size of between 10 and 30 nm.

2. The prosthesis of claim 1 wherein the first surface comprises features that are substantially spherical and about 20 nm in size.

3. The prosthesis of claim 1 further comprising a layer of mammalian cells on the first surface.

4. The prosthesis of claim 1 further comprising a layer of human chondrocytes on the first surface.

5. The prosthesis of claim 1 wherein the prosthesis is affixed to bone tissue in a human patient.

6. The prosthesis of claim 1 wherein human cartilage is affixed to the first surface.

7. The prosthesis of claim 6 wherein the alloy further comprises molybdenum.

8. The prosthesis of claim 1 wherein the prosthesis is a knee prosthesis.

9. A prosthesis for supporting the growth of mammalian cells, the prosthesis comprising a cobalt chromium alloy surface having a surface energy of greater than 30 mJ/m².

10. The prosthesis of claim 9 wherein human chondrocytes are disposed on the surface.

11. The prosthesis of claim 9 further comprising molybdenum.

12. The prosthesis of claim 9 having a portion consisting essentially of a cobalt chromium alloy.

13. The prosthesis of claim 9 having a portion consisting essentially of a cobalt chromium alloy doped with molybdenum.

14. The prosthesis of claim 9 comprising a screw for insertion into bone.

15. The prosthesis of claim 14 comprising a first portion including a contoured surface of cobalt chromium alloy and a second portion including a titanium screw.

16. A prosthesis for supporting mammalian cells, the prosthesis comprising a contoured surface comprising a cobalt chromium alloy exhibiting a wet contact angle of less than about 60 degrees.

17. The prosthesis of claim 16 wherein the wet contact angle is less than about 45 degrees.

18. The prosthesis of claim 16 wherein the wet contact angle is less than about 20 degrees.
